(19) United States
(12) Patent Application Publication
(10) Pub. No.: US 2021/0015776 A1
(43) Pub. Date: Jan. 21, 2021

(54) METHODS OF STABILIZATION OF LEVOTHYROXINE SODIUM TABLETS

(71) Applicant: Ascent Pharmaceuticals, Inc., Hauppauge, NY (US)

(72) Inventors: Sridhar Gumudavelli, Stony Brook, NY (US); Mithinti Naga Murali Krishna, Hyderabad (IN); Arjuna Rao, Hyderabad (IN); Meghana Vidiyala, Glen Head, NY (US); Sudhakar Vidiyala, Glen Head, NY (US)

(21) Appl. No.: 16/511,494

(22) Filed: Jul. 15, 2019

(51) Int. Cl. A61K 31/198; A61K 47/10; A61K 9/20; A61J 1/03; A61K 47/22

(52) U.S. Cl. CPC A61K 31/198; A61K 47/10; A61K 47/22; A61J 1/03; A61J 1/035; A61K 9/20

(57) ABSTRACT

The disclosure describes methods which substantially increase the stability of levothyroxine sodium. The use of antioxidants sufficient to inhibit oxidative degradation are used in the pharmaceutical composition of tablets, which include a therapeutically effective amount of levothyroxine sodium. Packaging systems, including high-density polyethylene (HDPE) bottles, child-resistant caps (CRC) of HDPE Bottles, and blister packs, incorporate antioxidants, oxygen scavengers and/or desiccants that are capable of establishing and maintaining arid and anaerobic conditions for packaging of Levothyroxine Sodium Tablets.

### BACKGROUND OF THE INVENTION

The present invention describes methods which substantially increase the stability of levothyroxine sodium. The use of antioxidants sufficient to inhibit oxidative degradation are used in the pharmaceutical composition of tablets, which include a therapeutically effective amount of levothyroxine sodium. Packaging systems, including high-density polyethylene (HDPE) bottles, child-resistant caps (CRC) of HDPE Bottles, and blister packs, incorporate antioxidants, oxygen scavengers and/or desiccants that are capable of establishing and maintaining arid and anaerobic conditions for packaging of Levothyroxine Sodium Tablets.

All references cited herein are incorporated herein by reference in their entireties.

### BRIEF SUMMARY

The disclosure provides a storage stable pharmaceutical tablet comprising an active agent which is sensitive to oxygen comprising: A therapeutically effective amount of a thyroxine active drug; An amount of an antioxidant sufficient to inhibit oxidative degradation of active drug. The disclosure provides a tablet wherein said thyroxine active drug is levothyroxine sodium. The disclosure provides a tablet wherein said antioxidant is selected from a group consisting of butyl hydroxyl anisole (BHA), butyl hydroxyl toluene (BHT), Vitamin C, and combinations thereof. The disclosure provides a tablet wherein said thyroxine active drug is comprised of levothyroxine sodium between 0.025 mg to about 0.3 mg The disclosure provides a tablet wherein said amount of antioxidant sufficient to inhibit oxidative degradation of thyroxine active drug is 0.01% of total weight. The disclosure provides a tablet wherein said amount of antioxidant sufficient to inhibit oxidative degradation of thyroxine active drug is 0.05% of total weight. The disclosure provides a tablet wherein said amount of antioxidant sufficient to inhibit oxidative degradation of thyroxine active drug is 0.1% of total weight. The disclosure provides a tablet wherein said amount of antioxidant sufficient to inhibit oxidative degradation of thyroxine active drug is 0.15% of total weight. The disclosure provides a tablet wherein said amount of antioxidant sufficient to inhibit oxidative degradation of thyroxine active drug is 0.2% of total weight. The disclosure provides a tablet wherein said amount of antioxidant sufficient to inhibit oxidative degradation of thyroxine active drug is 0.25% of total weight. The disclosure provides a tablet wherein said amount of antioxidant sufficient to inhibit oxidative degradation of drug is 0.3% of total weight. The disclosure provides a composition wherein said antioxidant is BHA. The disclosure provides a storage stable pharmaceutical tablet comprising an active agent that is sensitive to oxygen comprising: A therapeutically effective amount of a thyroxine active drug; an amount of an antioxidant sufficient to inhibit oxidative degradation of active drug. The disclosure provides a tablet wherein said thyroxine active drug is levothyroxine sodium. The disclosure provides a tablet wherein said antioxidant is selected from a group consisting of BHA, BHT and Vitamin C. The disclosure provides a tablet wherein said thyroxine active drug is comprised of levothyroxine sodium between 0.025 mg to about 0.3 mg. The disclosure provides a tablet wherein said amount of antioxidant sufficient to inhibit oxidative degradation of thyroxine active drug is 0.01% of total weight. The disclosure provides a tablet wherein said amount of antioxidant sufficient to inhibit oxidative degradation of thyroxine active drug is 0.05% of total weight. The disclosure provides a tablet wherein said amount of antioxidant sufficient to inhibit oxidative degradation of thyroxine active drug is 0.1% of total weight. The disclosure provides a tablet wherein said amount of antioxidant sufficient to inhibit oxidative degradation of thyroxine active drug is 0.15% of total weight. The disclosure provides a tablet wherein said amount of antioxidant sufficient to inhibit oxidative degradation of thyroxine active drug is 0.2% of total weight. The disclosure provides a tablet wherein said amount of antioxidant sufficient to inhibit oxidative degradation of thyroxine active drug is 0.25% of total weight. The disclosure provides a tablet wherein said amount of antioxidant sufficient to inhibit oxidative degradation of thyroxine active drug is 0.3% of total weight. The disclosure provides a composition wherein said antioxidant is BHT. The disclosure provides a storage stable pharmaceutical tablet comprising an active agent that is sensitive to oxygen comprising: a therapeutically effective amount of a thyroxine active drug; An amount of an antioxidant sufficient to inhibit oxidative degradation of active drug. The disclosure provides a tablet wherein said thyroxine active drug is levothyroxine sodium. The disclosure provides a tablet wherein said antioxidant is selected from a group consisting of BHA, BHT and Vitamin C. The disclosure provides a tablet wherein said active drug is comprised of levothyroxine sodium between 0.025 mg to about 0.3 mg. The disclosure provides a tablet wherein said amount of antioxidant sufficient to inhibit oxidative degradation of thyroxine active drug is 0.01% of total weight. The disclosure provides a tablet wherein said amount of antioxidant sufficient to inhibit oxidative degradation of thyroxine active drug is 0.05% of total weight. The disclosure provides a tablet wherein said amount of antioxidant sufficient to inhibit oxidative degradation of thyroxine active drug is 0.1% of total weight. The disclosure provides a tablet wherein said amount of antioxidant sufficient to inhibit oxidative degradation of thyroxine active drug is 0.15% of total weight. The disclosure provides a tablet wherein said amount of antioxidant sufficient to inhibit oxidative degradation of thyroxine active drug is 0.2% of total weight. The disclosure provides a tablet, wherein said amount of antioxidant sufficient to inhibit oxidative degradation of thyroxine active drug is 0.25% of total weight. The disclosure provides a tablet wherein said amount of antioxidant sufficient to inhibit oxidative degradation of thyroxine active drug is 0.3% of total weight. The disclosure provides a composition wherein said antioxidant is Vitamin C. The disclosure provides a storage stable pharmaceutical tablet comprising an active agent that is sensitive to oxygen comprising: A therapeutically effective amount of a thyroxine active drug; an amount of an antioxidant sufficient to inhibit oxidative degradation of active drug. The disclosure provides a tablet wherein said thyroxine active drug is levothyroxine sodium. The disclosure provides a tablet wherein said antioxidants are selected from a group consisting of BHA, BHT and Vitamin C. The disclosure provides a tablet wherein said active drug is comprised of levothyroxine sodium between 0.025 mg to about 0.3 mg. The disclosure provides a tablet wherein said amount of antioxidants sufficient to inhibit oxidative degradation of thyroxine active drug is 0.005% and 0.005% of total weight. The disclosure provides a tablet wherein said amount of antioxidants sufficient to inhibit oxidative degradation of thyroxine active drug is 0.025% and 0.025% of total weight. The disclosure provides a tablet wherein said amount of antioxidants sufficient to inhibit oxidative degradation of thyroxine active drug is 0.05% and 0.05% of total weight. The disclosure provides a tablet wherein said amount of antioxidants sufficient to inhibit oxidative degradation of thyroxine active drug is 0.075% and 0.075% of total weight. The disclosure provides a tablet wherein said amount of antioxidants sufficient to inhibit oxidative degradation of thyroxine active drug is 0.1% and 0.1% of total weight. The disclosure provides a tablet wherein said amount of antioxidants sufficient to inhibit oxidative degradation of thyroxine active drug is 0.125% and 0.125% of total weight. The disclosure provides a tablet wherein said amount of antioxidants sufficient to inhibit oxidative degradation of thyroxine active drug is 0.15% and 0.15% of total weight. The disclosure provides a composition wherein said antioxidants are BHA and BHT in equal amounts. The disclosure provides a packaging bottle for a pharmaceutical product comprising an active agent that is sensitive to oxygen comprising: A high density polyethylene bottle comprising: Antioxidants sufficient to inhibit oxidative degradation. The disclosure provides a package wherein the pharmaceutical product contains an active agent comprising of levothyroxine sodium. The disclosure provides a package, wherein said antioxidants are BHA, BHT and Vitamin C.

The disclosure provides a packaging cap for a pharmaceutical product comprising an active agent that is sensitive to oxygen and/or moisture comprising: A Child-Resistant Cap of a High Density Polyethylene Bottle comprising: An oxygen scavenger and/or a desiccant. The disclosure provides a package wherein the pharmaceutical product contains an active agent comprising of levothyroxine sodium. The disclosure provides a package wherein the oxygen scavenger is selected from the group consisting of organic, inorganic, metallic, non-metallic and enzymatic in nature. The disclosure provides a package where in the desiccants is selected from the group consisting of organic desiccants, inorganic desiccants, uncoated polymers, coated adsorbent polymers, and combinations thereof. The disclosure provides a packaging system for a pharmaceutical product comprising an active agent that is sensitive to oxygen comprising: A blister pack comprising: a blister sheet comprising of an array of interconnected cavities connected through channels that allows the exchange of air between them; wherein one or more of the cavities serve as a reservoir holding an oxygen scavenger; Further wherein one or more of the cavities comprising at least one pharmaceutical product that is sensitive to oxygen; A frangible lidding sealed to the sheet protecting the product in the cavity. The disclosure provides a package wherein the pharmaceutical product contains an active agent that comprises of levothyroxine sodium. The disclosure provides a package wherein the blister sheet has light and moisture protectant properties. The disclosure provides a package wherein the blister sheet is made of materials selected from the group consisting of polyvinylchloride, polyvinylidene chloride, polycarbonate, polyester, copolyester, acrylonitrile, low density polyethylene, polypropylene, and combinations thereof. The disclosure provides a package wherein the oxygen scavenger is selected from the group consisting of organic, inorganic, metallic, non-metallic, and enzymatic in nature. The disclosure provides a package wherein the frangible lidding has moisture barrier properties.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### BRIEF DESCRIPTION OF SEVERAL VIEWS OF THE DRAWINGS The invention will be described in conjunction with the following drawings in which like reference numerals designate like elements and wherein:

FIG. 1 is a picture showing a Blister Pack.

FIG. 2 is a picture showing Desiccant in Child Resistant Cap (CRC),

FIG. 3 is a diagram showing an HDPE Bottle with Stabilizers.

### DETAILED DESCRIPTION

As used herein, the term “pharmaceutically acceptable salts” includes acid addition salts or addition salts of free bases. The term “pharmaceutically acceptable salts” of a compound of the invention is also meant to include within its scope all the possible isomers and their mixtures, and any pharmaceutically acceptable metabolite, bioprecursor and/or pro-drug, such as, for example, a compound which has a structural formula different from the one of the compounds of the invention, and yet is directly or indirectly converted in vivo into a compound of the invention, upon administration to a subject, such as a mammal, particularly a human being.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may easily be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the term “agent” refers to any molecule, compound, methodology and/or substance for use in the prevention, treatment, management and/or diagnosis of a disease or condition. As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” or “active agent” refers to any molecule, compound, and/or substance that is used for the purpose of treating and/or managing a disease or disorder.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, such as pain, emesis, and anorexia and/or the amelioration of one or more symptoms thereof resulting from the administration of one or more therapies. In certain embodiments, the treatment includes a step of selecting a patient in need of treatment.

As used herein, the term “container” refers to any storage or sealable means capable of containing substances or objects, and may include hard vessels, including canisters bottles or jars, or soft vessels, including bags.

As referred to herein, a “desiccant” is any material or compound which can remove moisture from the interior of a closed package or vessel either by reacting or combining with the entrapped moisture, and which preferably yields one or more innocuous products.

The term “derivative” or “derivatized” as used herein includes chemical modification of a compound of the invention, or pharmaceutically acceptable salts thereof or mixtures thereof. That is, a “derivative” may be a functional equivalent of a compound of the invention, which is capable of inducing the improved pharmacological functional activity in a given subject. As used herein, the terms “composition” and “formulation” are used interchangeably.

### Active Agent

Thyroxine active drugs are known for both therapeutic and prophylactic treatment of thyroid disorders. The thyroid accomplishes its regulation functions by producing the hormones L-triiodothyronine (liothyronine; T3) and L-thyroxine (levothyroxine; T4). The physiological actions of thyroid hormones are produced predominantly by T3, the majority of which (approximately 80%) is derived from T4 by deiodination in peripheral tissues.

The disclosure provides pharmaceutical compositions, and methods of preparing and administering such compositions, comprising the active agent Levothyroxine, Levothyroxine Sodium, and other pharmaceutical salts of Levothyroxine. Levothyroxine Sodium is a synthetic form of thyroid hormone Thyroxine, which is secreted from follicular cells of thyroid gland. Levothyroxine is ideally used for the treatment of thyroid hormone deficiencies such as hypothyroidism. Due to its ability to lower thyroid-stimulating hormone, Levothyroxine is also used in the treatment of goiter and also to prevent the recurrence of thyroid cancer.

Levothyroxine Sodium has the following chemical structure.

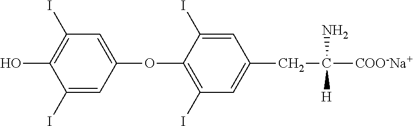

Administration of levothyroxine sodium provides T4 to a patient. Once absorbed, the administered T4 behaves identically to T4 that otherwise would be secreted by the thyroid gland of the patient, and binds to the same serum proteins, providing a supply of circulating T4-thyroglobulin in the patient. The administered T4 may be deiodinated in vivo to T3. As a result, a patient receiving appropriate doses of levothyroxine sodium will exhibit normal blood levels of T3, even when the patient's thyroid gland has been removed or is not functioning.

Levothyroxine sodium is prescribed for thyroid hormone replacement therapy in cases of reduced or absent thyroid function e.g., ailments such as myxedema, cretinism and obesity. Levothyroxine sodium is quite unstable, hygroscopic and degrades rapidly when subjected to high humidity, light or high temperature. Because of the physicochemical properties of the drug, formulations of levothyroxine sodium have extremely short stability duration, worsened under conditions of high humidity and temperature.

Levothyroxine sodium is available in the form of capsules, tablets and parenteral dosage forms.

A solid composition that includes levothyroxine sodium and mannitol may include from 25 to 1,000 microgram levothyroxine sodium. Preferably the composition includes from 50 to 750 microgram levothyroxine sodium, or from 100 to 500 microgram levothyroxine sodium. The amount of levothyroxine sodium in the composition may be an amount sufficient for a single initial dose of levothyroxine sodium, an amount sufficient for a single 2.sup.nd day dose of levothyroxine sodium, or an amount sufficient for a daily maintenance dose of levothyroxine sodium. The amount of levothyroxine sodium in the composition may be a different therapeutic amount. For example, the amount of levothyroxine sodium in the composition may be an amount sufficient for half of a single initial dose, half of a single 2.sup.nd day dose, or half of a daily maintenance dose. Presently preferred amounts of levothyroxine sodium in the composition include about 100 microgram, about 200 microgram, and about 500 microgram.

In exemplary embodiments, formulations as disclosed herein may comprise active agent at a concentration of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, In exemplary embodiments, formulations as disclosed herein may comprise active agent at a concentration of about 1 to about 20%, of about 5% to about 25%, about 10% to about 20%, or about 15% to about 18%, about 30% to about 70%, about 35% to about 65%, about 63.13%, and about 40% to about 64% w/w.

In an exemplary formulation as disclosed herein, the active agent will represent approximately 1 wt % to 75 wt %, preferably 2 wt % to 30 wt %, more preferably 5 wt. % to 20 wt. % of the total weight.

In other embodiments, the pharmaceutical compositions further comprise one or more additional materials such as a pharmaceutically compatible carrier, binder, viscosity modifier, filling agent, suspending agent, flavoring agent, sweetening agent, disintegrating agent, surfactant, preservative, lubricant, colorant, diluent, solubilizer, moistening agent, stabilizer, wetting agent, anti-adherent, parietal cell activator, anti-foaming agent, antioxidant, chelating agent, antifungal agent, antibacterial agent, or one or more combination thereof.

### Pharmaceutical Dosage Forms

The compounds and compositions of the present invention can be processed by agglomeration, air suspension chilling, air suspension drying, balling, coacervation, coating, comminution, compression, cryopelletization, encapsulation, extrusion, wet granulation, dry granulation, homogenization, inclusion complexation, lyophilization, melting, microencapsulation, mixing, molding, pan coating, solvent dehydration, sonication, spheronization, spray chilling, spray congealing, spray drying, or other processes known in the art. The compositions can be provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a buccal or sublingual solid, a granule, a film, a sprinkle, a pellet, a bead, a pill, a powder, a triturate, a platelet, a strip or a sachet. Compositions can also be administered as a “dry syrup”, where the finished dosage form is placed directly on the tongue and swallowed or followed with a drink or beverage. These forms are well known in the art and are packaged appropriately. The compositions can be formulated for oral, nasal, buccal, ocular, urethral, transmucosal, vaginal, topical or rectal delivery.

The pharmaceutical composition can be coated with one or more enteric coatings, seal coatings, film coatings, barrier coatings, compress coatings, fast disintegrating coatings, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance The definitions of these terms are known to those skilled in the art. In addition, the dosage form release profile can be affected by a polymeric matrix composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be effected through favorable diffusion, dissolution, erosion, ion-exchange, osmosis or combinations thereof.

When formulated as a capsule, the capsule can be a hard or soft gelatin capsule, a starch capsule, or a cellulosic capsule. Although not limited to capsules, such dosage forms can further be coated with, for example, a seal coating, an enteric coating, an extended release coating, or a targeted delayed release coating. These various coatings are known in the art, but for clarity, the following brief descriptions are provided: seal coating, or coating with isolation layers: Thin layers of up to 20 microns in thickness can be applied for variety of reasons, including for particle porosity reduction, to reduce dust, for chemical protection, to mask taste, to reduce odor, to minimize gastrointestinal irritation, etc. The isolating effect is proportional to the thickness of the coating. Water soluble cellulose ethers are preferred for this application. HPMC and ethyl cellulose in combination, or Eudragit E100, may be particularly suitable for taste masking applications. Traditional enteric coating materials listed elsewhere can also be applied to form an isolating layer.

Extended release coatings are designed to effect delivery over an extended period of time. The extended release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended release dosage forms can be readily designed by one skilled in art to achieve delivery to both the small and large intestines, to only the small intestine, or to only the large intestine, depending upon the choice of coating materials and/or coating thickness.

Enteric coatings are mixtures of pharmaceutically acceptable excipients which are applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: 1. resistance to dissolution and disintegration in the stomach; 2. impermeability to gastric fluids and drug/carrier/enzyme while in the stomach; 3. ability to dissolve or disintegrate rapidly at the target intestine site; 4. physical and chemical stability during storage; 5. non-toxicity; 6. easy application as a coating (substrate friendly); and 7. economical practicality.

Dosage forms of the compositions of the present invention can also be formulated as enteric coated delayed release oral dosage forms, i.e., as an oral dosage form of a pharmaceutical composition as described herein which utilizes an enteric coating to affect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

Delayed release generally refers to the delivery so that the release can be accomplished at some generally predictable location in the lower intestinal tract more distal to that which would have been accomplished if there had been no delayed release alterations. The preferred method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers for use in the present invention are anionic carboxylic polymers.

Shellac, also called purified lac, a refined product obtained from the, resinous secretion of an insect. This coating dissolves in media of pH>7. Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method of the present invention, the combination of the invention may be administered to mammalian species, such as dogs, cats, humans, etc. and as such may be incorporated in a conventional systemic dosage form, such as a tablet, capsule, elixir or injectable. The above dosage forms will also include the necessary carrier material, excipient, lubricant, buffer, antibacterial, bulking agent (such as mannitol), anti-oxidants (ascorbic acid of sodium bisulfite) or the like.

The dose administered must be carefully adjusted according to age, weight and condition of the patient, as well as the route of administration, dosage form and regimen and the desired result.

The pharmaceutical compositions of the invention may be administered in the dosage forms in single or divided doses of one to four times daily. It may be advisable to start a patient on a low dose combination and work up gradually to a high dose combination.

Liquid formulations can be prepared by dissolving or suspending one or the combination of active substances in a conventional liquid vehicle acceptable for pharmaceutical administration so as to provide the desired dosage in one to four teaspoonful.

Dosage forms can be administered to the patient on a regimen of, for example, one, two, three, four, five, six, or other doses per day

In order to more finely regulate the dosage schedule, the active substances may be administered separately in individual dosage units at the same time or carefully coordinated times. Since blood levels are built up and maintained by a regulated schedule of administration, the same result is achieved by the simultaneous presence of the two substances. The respective substances can be individually formulated in separate unit dosage forms in a manner similar to that described above.

In formulating the compositions, the active substances, in the amounts described above, may be compounded according to accepted pharmaceutical practice with a physiologically acceptable vehicle, carrier, excipient, binder, preservative, stabilizer, flavor, etc., in the particular type of unit dosage form.

Illustrative of the adjuvants which may be incorporated in the dosage form are the following: a binder such as gum tragacanth, acacia, corn starch or gelatin; an excipient such as dicalcium phosphate or cellulose; a disintegrating agent such as corn starch, potato starch, alginic acid or the like; a lubricant such as stearic acid or magnesium stearate; a sweetening agent such as sucrose, aspartame, lactose or saccharin; a flavoring agent such as orange, peppermint, oil of wintergreen or cherry. When the dosage unit form is a capsule, it may contain in addition to materials of the above type a liquid carrier such as a fatty oil. Various other materials may be present as coatings or to otherwise modify the physical form of the dosage unit. For instance, capsules may be coated with shellac, sugar or both. A syrup of elixir may contain the active compound, water, alcohol or the like as the carrier, glycerol as solubilizer, sucrose as sweetening agent, methyl and propyl parabens as preservatives, a dye and a flavoring such as cherry or orange.

The liquid formulations useful herein may comprise a solvent, solution, suspension, microsuspension, nanosuspension, emulsion, microemulsion, gel or even a melt containing the active component or components. In some embodiments the nanoparticles, nanofibers, or nanofibrils may be in the form of, or within or on, granules, powders, suspensions, solutions, dissolvable films, mats, webs, tablets, or releasable forms particularly releasable dosage forms. Other particular useful forms are concentrates to which a diluting liquid is added prior to use. The product may also be sprayed onto the inner surface of a container to which a liquid is added later prior to use and the nanoparticles, nanofibers, or nanofibrils, are released into the liquid. Pharmaceutical compositions of the present invention can include nanoparticles, composite nanoparticles, nanosuspension, or nanocapsules of the present invention.

A dose may be administered in a single dosage form or in multiple dosage forms. When multiple dosage forms are used the amount of compound contained within each dosage form may be the same or different. The amount of a composition of the invention contained in a dose may depend on the route of administration and whether the disease in a patient is effectively treated by acute, chronic, or a combination of acute and chronic administration.

In certain embodiments an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a composition of the invention may exhibit a high therapeutic index. The data obtained from cell culture assays and animal studies may be used in formulating a dosage range that is not toxic for use in, for example, humans. A dose of a composition of the invention provided by the present disclosure may be within a range of circulating concentrations in for example the blood, plasma, or central nervous system, that include the effective dose and that exhibits little or no toxicity. A dose may vary within this range depending upon the dosage form employed and the route of administration utilized. In certain embodiments, an escalating dose may be administered.

### Stabilizers

In certain preferred embodiments, the formulation contains amounts of one or more pharmaceutically acceptable stabilizer, such as antioxidants, in an amount effective to stabilize the active pharmaceutic ingredient contained therein such that the active pharmaceutic ingredient does not degrade to an unacceptable extent and the formulation is deemed stable as per the ICH guidance for two-year expiration dating when placed under storage conditions selected from (i) 25° C./60% relative humidity (RH) for 12 months; (ii) 30° C./60% relative humidity (RH) for 6 months; (iii) 40° C./60% relative humidity (RH) for 6 months; and (iv) any combination thereof.

In further embodiments of the invention, an effective stabilizing amount of one or more pharmaceutically acceptable stabilizer such as an antioxidants is added to the formulation. The term “anti-oxidant” is used herein to describe any compound which is oxidized more easily than the active pharmaceutic ingredient compounds included in the dosage forms of the present invention. Any of the known anti-oxidants may be used, including but not limited to anti-oxidants such as butyl hydroxyl anisole (BHA), butyl hydroxyl toluene (BHT), propyl gallate, lecithin, Vitamin E tocopherol, sesamin, sesamol, sesamolin, alpha tocopherol, ascorbic acid, ascorbyl palmitate, fumaric acid, malic acid, sodium ascorbate and sodium metabisulphite, as well as chelating agents such as disodium EDTA, may also be used to stabilize the active pharmaceutic ingredient formulations of the present invention.

The preparation may also contain anti-oxidant synergists to prevent oxidative degradation. Any of the known anti-oxidant synergists may also be used in effective amounts, for example disodium edetate.

The amount of stabilizer, such as an antioxidant, which may be used will be optimized for each formulation, in order to obtain a stable product (dosage form) having the desired shelf-life. Generally speaking, in embodiments in which an anti-oxidant is included, suitable formulations may include from about 0.001% to about 20% w/w of a pharmaceutically acceptable anti-oxidant(s). For example, in certain preferred embodiments, the amount of lecithin included in the active pharmaceutic ingredient dosage form is in the range from about 0.1 to about 10% w/w, and in certain embodiments more preferably from about 0.3% to about 8.25% w/w. In other preferred embodiments, the amount of L-ascorbic acid-6-palmitate is from about 0.001 to about 1%, w/w, and in certain embodiments more preferably in the range from about 0.01% to about 0.1% w/w. The anti-oxidant preferably prevents the formation of degradants in the dosage form such as those mentioned herein.

### Antioxidant

“Antioxidants” include, e.g., butylated hydroxytoluene (BHT), sodium ascorbate, ascorbic acid (vitamin C), tocopherol, ascorbic acid, ascorbyl palmitate, sodium ascorbate, butylated hydroxyanisole (BHA), butylated hydroxytoluene (BHT), propyl gallate, malic acid, fumaric acid, potassium metabisulfite, sodium bisulfite, sodium metabisulfite, and tocopherols, e.g., α-tocopherol (vitamin E)). For example, antioxidants such as BHA and/or BHT may each be present in an amount of from about 0.01% to about 0.750% by weight based upon the total weight of the composition, e.g., about 0.05% to about 0.35% by weight, e.g., at least about 0.01, 0.05, 0.10, 0.15, 0.20, 0.25, 0.30, 0.35, 0.40, 0.45, 0.50%, 0.60%, 0.65%, 0.70%, 0.75% by weight.

### Viscosity Modifier

In certain embodiments, the composition and methods as disclosed herein may comprise at least one viscosity modifier. In certain embodiments, the viscosity modifier may be a binder. Binders are used to impart cohesive qualities to compositions and methods as disclosed herein, and thus ensure that the compositions and methods as disclosed herein remains intact. Suitable viscosity modifiers and/or binder materials include, but are not limited to, starch (including corn starch and pregelatinized starch), gelatin, sugars (including sucrose, glucose, dextrose and lactose), polyethylene glycol, waxes, yellow wax, and natural and synthetic gums, e.g., acacia sodium alginate, polyvinylpyrrolidone (povidone; PLASDONE®), cellulosic polymers (including hydroxypropyl cellulose, hydroxypropyl methylcellulose, methyl cellulose, microcrystalline cellulose, ethyl cellulose, hydroxyethyl cellulose, and the like), and combinations thereof. In certain embodiments, the viscosity modifier may be a lubricant. Lubricants are used to facilitate tablet manufacture, promoting powder flow and preventing particle capping (i.e., particle breakage) when pressure is relieved. Useful viscosity modifiers and/or lubricants include, but are not limited to, magnesium stearate, calcium stearate, stearic acid, and hydrogenated vegetable oil (preferably comprised of hydrogenated and refined triglycerides of stearic and palmitic acids at about 1 wt. % to 5 wt. %, most preferably less than about 2 wt. %). Non-limiting examples of suitable viscosity modifiers and/or lubricants include talc, magnesium stearate, calcium stearate, zinc stearate, colloidal silicon dioxide, hydrogenated vegetable oils, Hydrogenated Vegetable Oil Type I, Hydrogenated Vegetable Oil Type II, polyoxyethylene monostearate, polyethylene glycol, sodium stearyl fumarate, sodium benzoate, sodium lauryl sulfate, magnesium lauryl sulfate, light mineral oil, and combinations thereof. In exemplary embodiments, the lubricant may be magnesium stearate.

In embodiments in which the lubricant is included in the compositions and methods as disclosed herein, the amount of the lubricant may range from about 0.1% to about 3% by weight of the pharmaceutical composition. In various embodiments, the amount of the lubricant may range from about 0.1% to about 0.3%, from about 0.3% to about 1%, or from about 1% to about 3% by weight of the pharmaceutical composition. In exemplary embodiments, the amount of the lubricant may be about 0.5%, about 1%, about 1.5%, about 2%, about 2.5%, about 3%, about 3.5%, about 4%, about 4.5%, or about 5% by weight of the pharmaceutical composition.

Viscosity modifiers may be present in a concentration of, for example, from about 0.25 wt. % to about 3 wt. %, 0.5 wt. % to about 2.0 wt. %, from about 0.75% to about 1.5%, from about 0.5% to about 12%, from about 1% to about 11%, from about 2% to about 10%, about 1%, about 2%, about 3%, about 4%, about 5%, about 6%, about 7%, about 8%, about 9%, about 10%, about 11%, about 12%, about 15%, about 14%, about 15%, and about 16%.

Other viscosity modifiers and/or binders include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

### Sweetening Agents

In further embodiments, the present invention may comprise a sweetening agent. Suitable sweetening agents include, but are not limited to, sucralose, sucrose, aspartame, saccharin, dextrose, mannitol, xylitol, ethyl maltol, saccharin sodium, dipotassium glycyrrhizinate, stevia, thaumatin, acesulfame K, and combinations thereof. If the formulations contain a sweetener, the formulations preferably contain from about 0.001 to about 1% sweetener.

### Packaging System

The deoxygenated, desiccated or deoxygenated and desiccated oxidation-sensitive material may be packaged in any appropriate package form which can maintain a substantially oxygen/moisture-free environment for a prolonged period, and thus form a barrier to external oxygen/moisture such as, for example, sachets, bags, bottles (glass or plastic), deep drawn packages or blister packs. For example, single dose amounts of free oxidation-sensitive material may be sealed in sachets, or bags, or dosage forms provided as single dosage units or multiple dosage units to be used in a single day may be sealed in small bags or bottles. Blister packs provide the advantage of protecting the product from outer influences while enabling the deliberate and controllable removal of single dosage units at the desired time of intake. In accordance with the present invention, the composition of the disclosure is placed into a container, along with an oxygen-scavenging material. In certain embodiments, a moisture absorber is also added to the container. The moisture absorber can be added as a separate component, or may be incorporated with the oxygen scavenging material. In certain embodiments, the moisture absorber can be a desiccant.

Containers used in the present invention can be any packaging container suitable to house pharmaceutical formulations. Examples of containers which may be used in the present invention include, e.g., bottles, vials, blister packs, foil packs, pouches, bulk containers, single dose containers, multidose containers and the like. The containers may be made, e.g., of plastic or glass, and may be clear, colored, tinted, coated, etc. In certain embodiments, the containers are made with high density polyethylene (HDPE), which provides a moisture barrier to the contents.

Containers used in the present invention may be used in conjunction with any suitable type of closure known in the art, such as, e.g., rubber closures, screw caps, crown caps, snap-on closures, friction-fit closures, tamper evident seals, dispensing closures, child-resistant closures, and any combination thereof In certain embodiments, the closure will prevent any excess oxygen from entering the container when secured onto the container.

Preferably, the packaging system of the present invention provides stability of the composition of the disclosure at room temperature for at least 3 months, at least 6 months, at least 12 months, at least 18 months, at least 24 months, at least 30 months, or at least 36 months. Preferably, the packaging system of the present invention provides a room temperature stable composition of the disclosure when measured at 3 months, at 6 months, at 12 months, at 18 months, at 24 months, at 30 months, or at 36 months.

The term Blister packs herein is taken to mean packaging comprising at least two sheets, films, or foils which are firmly bonded to one another and which contain cavities for the accommodation of the pharmaceutical product to be packed. Blister packs usually consist of a thermoformed plastic sheet or film (cavity sheet, blister sheet, or shaped film) for the accommodation of the solids, which, after filling, is firmly bonded, e.g., heat-sealed, to a second sheet, film or foil (cover sheet, frangible layer, film or foil), which usually consists of an aluminium foil and/or plastic sheet or film. The packaged pharmaceutical product s can be pushed through the cover sheet, frangible layer, film or foil by pressure on the cavity sheet, film or foil and removed individually from the blister pack. Blister packs are therefore also known as push-through packs. If “pushing-through” the cover sheet, film or foil is not possible owing to the shape, size and/or strength of the solids contained, the blister packs can also be opened by slitting open the cover sheet, film or foil using a sharp object, for example using the finger nail. However, the term “blister pack” is not restricted thereto, but also encompasses special embodiments, such as, for example, child-proof modifications, such as, for example, those in which it is necessary to carry out two different opening operations whose sequences are intended to be beyond the intellectual capacity of children (such as so-called “peel-push systems”), or embodiments in which the cover sheet, film or foil is not punctured, but instead peeled off before removal of the solids contained.

Blister packs are the preferred primary packaging means for solid pharmaceutical administration forms. Advantages are that the administration forms can be removed individually and thus without contamination of the other administration forms, which are furthermore contained in sealed cavities, the administration forms are separated from one another (meaning that mutual interaction, such as, for example, abrasion or sticking, are basically prevented).

A further important function of blister packs is protection of the pharmaceutical administration forms contained therein against harmful environmental influences, such as light, gases, in particular oxygen, and against moisture. Since blister packs are usually accommodated in folding cartons, which are not effective barriers against moisture and gases, the crucial protective action in the case of solid pharmaceutical administration forms packaged in blister packs (primary packaging means) and folding cartons (secondary packaging means) arises through the blister packs.

Various plastic sheets or films, such as, for example, polyvinyl chloride (PVC), polyvinylidene chloride (PVDC), high-density polyethylene (HDPE), polypropylene (PP), polyethylene terephthalate (PET), polycarbonate, each of which have different material properties may be used in the blister packs of the invention. Although the selection of a material of relatively low permeability for moisture and/or oxygen or the use of composite sheets or films made from these materials, such as, for example, PVC/PVDC, PVC/HDPE, optionally also together with further polymers as barrier layer, such as, for example, cycloolefin copolymer (COC), or special polyhalogenated polymers, such as polychlorotrifluoroethylene (PCTFE) Aclar®, (PVC/PCTFE, PP/COC (for example Polybar®) PVC/COC/PVDC composite sheets or films), enables the ingress of moisture to be reduced to a certain degree, it does not prevent it completely.

Suitable for use for the manufacture of blister packs which are suitable for the pack according to the invention are all plastic sheets or films which can be converted into blister packs in corresponding plants, in particular thermoforming plants. Examples of plastic sheets and films which are suitable for the manufacture of blister packs are polyvinyl chloride (PVC), polyvinylidene chloride (PVDC), high-density polyethylene (HDPE), polypropylene (PP), polyethylene terephthalate (PET), polycarbonate, cycloolefin copolymer (COC), special polyhalogenated polymers, such as polychlorotrifluoroethylene (PCTFE) Aclar®, and composite sheets or films made from these materials, such as, for example, PVC/PVDC, PVC/HDPE, PVC/PCTFE, PP/COC (Polybar®) PVC/COC/PVDC, particularly suitable are PVC, PVDC, HDPE, PP, PET and composite sheets or films made from these, very particularly suitable PVC, PP, and PET. The plastic sheets or films can be employed as cavity sheet or film and/or as cover sheet or film. The cavity sheet or shaped film at least preferably consists of a plastic sheet or film.

Plastic sheets or films of low thickness are preferably and advantageously employed for the manufacture of the blister packs. The plastic sheets or films used as cavity sheet or film usually have a thickness of 10 to 500 □m, preferably 15 to 300 □m, particularly preferably 15 to 100 □m, very particularly preferably 15 to 50.

The blister pack may also include a frangible lidding, affixed to the shaped film, so that the single unit dose of the pharmaceutical product is substantially confined between the frangible lidding and the cavity. The frangible lidding is preferably made from aluminum foil sufficiently thin so as to enable a consumer to push the single unit dose through it by pressing on the underside of the cavity, or it may be made from an aluminum foil laminate (i.e., layers of aluminum, polyethylene terepthalate (PET) and/or paper) that is attached to the shaped film in a manner that permits the consumer to easily tear it away from each cavity, thereby gaining access to the single unit doses.

In an exemplary embodiment, the blister pack comprises a frangible lidding which is sealed or affixed to the shaped film such as by heat induction, for instance, so that the single unit doses of pharmaceutical product are substantially confined between the wells of the cavities and the frangible lidding.

A suitable blister pack, e.g. for a pharmaceutical composition or combination of the invention, comprises or is formed of a top foil or frangible layer (which is breachable by the tablets) and a bottom part (which contains pockets for the tablets). The top foil or frangible layer may contain a metalic foil, particularly an aluminium or aluminium alloy foil (e.g. having a thickness of 20 □m to 45 □m, preferably 20 □m to 25 □m) that is coated with a heat-sealing polymer layer on its inner side (sealing side). The bottom part may contain a multi-layer polymer foil (such as, e.g., poly(vinyl choride) (PVC) coated with poly(vinylidene choride) (PVDC); or a PVC foil laminated with poly(chlorotriflouroethylene) (PCTFE)) or a multi-layer polymer-metal-polymer foil (such as, e.g., a cold-formable laminated PVC/aluminium/polyamide composition).

Aluminium foil, which has low water permeability, is usually employed for sealing blister packs, such as in the frangible layer. Low water permeability is not necessary in the pack according to the invention, meaning that other materials can also be employed for sealing the blister packs. This enables the use of plastic sheets or films as cover sheet or film, where sheets or films made from the same material as the cavity sheet or film can also be used. Single-material packaging of this type is particularly advantageous since it can be recycled without prior separation of cavity sheet or film and cover sheet or film, which is particularly desired for environmental protection reasons. On use of plastic sheets or films as cover sheet or film, water vapour present in the cavities of the blister pack can also be removed through the cover sheet or film, which advantageously increases the drying rate of the pharmaceutical administration forms contained in the blister pack. If, in addition, a plastic sheet or film of very low material thickness is used, a further increase in the drying rate and easier removal of the solid pharmaceutical administration form contained in the blister pack arise, since this can be pushed through more easily, besides reduced material usage.

To ensure a long storage period especially under hot and wet climate conditions an additional overwrap or pouch made of a multi-layer polymer-metal-polymer foil (e.g. a laminated polyethylen/aluminium/polyester composition) may be used for the blister packs.

Blister cards can, for example, contain a foil backing as a barrier. Blister cards or packaging can include, for example, triplex blister film of different types, such as standard and high barrier films, including, for example, triplex Flexafarm Sbc (e.g., PVC 250 my+PE 25 my+PVDC 150 g/mq sbc grade) and Aquaba-PVC (e.g., PVC 250 my+AQUABA 160 g/mq), Aclar, Alu-Alu formats, triple layer blister foil (OPA) with soft tempered aluminium in central position, other layers PVC and polyamide, and new generation multilayer blister combined materials.

The article may further comprise a label or package insert, which refer to instructions customarily included in commercial packages of therapeutic products, that may contain information about the indications, usage, dosage, administration, contraindications and/or warnings concerning the use of such therapeutic products. In one embodiment, the label or package inserts indicates that the composition can be used for any of the purposes described herein.

For most situations, but not all, the blister pack will include a plurality of cavities, a plurality of airflow channels (at least one airflow channel per cavity), which together permit oxygen and moisture trapped in the plurality of cavities to easily pass out of the cavities, into the plurality of airflow channels and through the plurality of airflow channels to the reservoir comprising the absorbent. In some embodiments, however, there may even exist a plurality of airflow channels for every cavity in the blister.

Where there is a concern that the single unit doses of drugs inside the cavities in the blister pack may be too easily accessed by a child, embodiments of the invention may also include a hard plastic “shell pack” container, configured to receive, cover and protect the blister pack from direct access until the blister pack is extracted from inside the shell pack. In this alternative configuration, the blister pack may be inserted into the shell pack, and the shell pack sealed inside the sealed outer container during the package manufacturing stage.

### Oxygen Scavenging Materials

Oxygen scavenging materials used in accordance with the present invention may comprise oxygen scavenging substances. Suitable oxygen-scavenging substances comprise at least one material capable of reacting with or absorbing molecular oxygen, thereby limiting the amount of oxygen available for oxidative degradation. Preferably, materials are selected that do not react with oxygen so quickly that handling of the materials is impracticable. Therefore, stable oxygen-scavenging materials that do not readily explode or burn upon contact with molecular oxygen and are useful during extended shelf-life are preferred.

Oxygen scavengers that can be utilized in the present invention include those based on metal (e.g., organometallic ligands, iron, calcium, magnesium, scandium, titanium, vanadium, chromium, manganese, cobalt, nickel, copper, zinc, silver, tin, aluminum, antimony, germanium, silicon, lead, cadmium, rhodium or combinations thereof), sulfites, boron, glycols and sugar alcohols (e.g., catechol), oxidative enzymes (e.g., glucose oxidase), antioxidants (e.g., ascorbic acid), unsaturated fatty acids and hydrocarbons, palladium catalysts, yeast, photosensitive dyes, polyamides (e.g., polydiene block copolymers or polymer bound olefins), aromatic nylon, or any mixtures thereof.

The term “organic based scavenger” as used herein includes the following oxygen scavengers glycols, sugar alcohols (e.g. catechol), ascorbic acid, unsaturated fatty acids, hydrocarbons, photosensitive dyes or combinations thereof.

The term “non-organic based scavenger” as used herein includes the following oxygen scavengers based on metal (e.g., organometallic ligands, iron, calcium, magnesium, scandium, titanium, vanadium, chromium, manganese, cobalt, nickel, copper, zinc, silver, tin, aluminum, antimony, germanium, silicon, lead, cadmium, rhodium or combinations thereof), sulfites, boron, palladium catalysts, and combinations thereof.

The term “polymer based scavenger” as used herein includes the following oxygen scavengers polyamides (e.g., polydiene block copolymers or polymer bound olefins), aromatic nylon, and combinations thereof.

The term “enzyme based scavenger” as used herein includes the following oxygen scavengers oxidative enzymes such as oxidases, e.g. glucose oxidase, yeast, or combinations thereof.

The term “oxidative degradation” as used herein refers to the oxidation of the active agent to other components such as the degradation products listed above as impurities. Also contemplated by the present invention are containers that may comprise at least two or more oxygen scavenging materials, wherein each material has different oxygen scavenging properties.

The oxygen scavenging materials may be contained in a canister or packet/sachet that is placed into the contained which houses the active agent composition. In other embodiments, the oxygen scavenging materials may also be incorporated into the container which houses the active agent composition. For example, containers may be manufactured to contain the oxygen scavenging material within the container itself, as can be found in, e.g., Oxy-Guard® Barrier Bottles (available from Süd-Chemie AG), extrusion blow-molded six-layer pharmaceutical containers which incorporate oxygen scavenging materials within at least one of the six layers to provide against oxidation of the contents.

Commercially available oxygen scavenger materials include, e.g., FreshPax® sachets (available from Multisorb Technologies Inc), Ageless® Z (Ageless-Z is designated as Z-100, Z-1000, etc., to indicate the milliliters of oxygen with which a single packet will react), StabilOx® (available from Multisorb Technologies Inc), O-Busters® (available from Hsiao Sung Non-Oxygen Chemical Co., Ltd), Bioka Oxygen Absorber (available from Bioka Ltd.), PharmaKeep® (Types CH, KH and KD) and the like.

### Moisture Absorbing Materials

A desiccant is any drying agent that removes moisture from the air. Desiccants include, but are not limited to, silica gel, clay desiccants, calcium sulfate, calcium chloride, calcium oxide, zeolite, activated alumina, activated charcoal and combinations thereof. However, other vapor or moisture absorbing mechanisms are not beyond the scope of the present invention. Other vapor or moisture absorbing materials include desiccants made from inorganic materials such a zeolites and aluminas Such inorganic materials of vapour or moisture absorbing materials have high water absorption capacities and favourable water absorption isotherm shapes. The water absorption capacity of such materials typically varies from 20 to 50 weight percent. In the preferred embodiment, the absorbing material is a MINIPAX™ supplied by Multisorb Technologies in the United States and Silgelac in Europe (silica gel packaged inside TYVEK®, which is a nylon mesh bonded with a microporous polyurethane). Other exemplary moisture absorbing materials include, but are not limited to, alumina, bauxite, anhydrous, calcium sulphate, water-absorbing clay, activated bentonite clay, a molecular sieve, or other like materials which optionally include a moisture sensitive colour indicator such as cobalt chloride to indicate when the desiccant is no longer operable. While in the preferred embodiment of the present invention, the package is designed to substantially prevent ingression of water vapor and particulate matter into the enclosed volume, the moisture absorbing material is placed within the reservoir in order to absorb any residual moisture present in the atmosphere.

The desiccant should be present in an amount sufficient to absorb any residual moisture inside the package. Moreover, the desiccant should be present in an amount sufficient to absorb any moisture that possibly ingresses from the external environment. It is also possible to place the desiccant inside the reservoir.

Preferably the desiccant is selected from the group consisting of silica gel, zeolite, alumina, bauxite, anhydrous calcium sulphate, activated bentonite clay, water-absorbing clay, molecular sieve and any mixtures thereof.

Absorbents which may be present are in principle any type of desiccants, i.e. moisture-binding binders. Desiccants may include chemical substances which form hydrates with water. Examples of chemical substances of this type are anhydrous salts, which tend to absorb water or moisture and in the process form a stable hydrate. The moisture is bound and liberation thereof is prevented by a chemical reaction.

Desiccants may contain substances which are reactive. The substances react with water or moisture by forming a new substance. The newly formed substances are normally stable at low temperatures, which is only reversible with expenditure of high energy. Desiccants of this type are principally used for drying solvents and as water-absorbent material in the case of polymers which themselves have to remain in a reduced-moisture state.

Desiccants may bind the moisture by physical adsorption. The desiccant contains particles having fine capillaries into which the moisture is drawn. The pore size of the capillaries and the density thereof in the desiccant determine the absorption properties. Examples of desiccants of this type are molecular sieves, silica gels, certain synthetic polymers.

In further or alternative embodiments a desiccant or other such materials which are substantially absorbent to water vapor may accompany the vial and or sealed container within the outer packaging. Suitable materials for use as desiccants include oxides of aluminum, calcium, titanium, zirconium, silicon, thorium, magnesium and barium, alumina, alumina hydrates, natural and synthetic molecular sieves, silica gel, precipitated silica, clays, perchlorates, zeolite, natural gums, magnesium or calcium sulfate, calcium, lithium or cobalt chloride, and calcium carbonate. An indicator dye can also be added to the desiccant material to provide for monitoring the amount of moisture absorbed during storage of the product package.

The amount of desiccant that may used will depend on several factors including the moisture permeability of the types of materials used in making the product package, the moisture absorbing capacity of the particular desiccant material, and the intended shelf life of the drug. The minimal amount to be used is that amount that will effectively absorb water vapor within the product package over the intended shelf life of the drug, typically three years, and achieve an acceptable level of drug loss from crystallization or degradation to still deliver a therapeutically effective amount of the drug. The desiccant should be capable of absorbing at least about 1.5 grams to about 5 grams of moisture over the intended period of storage and use of the product package. The amount of desiccant material needed to prevent such moisture contamination can be determined by one skilled in the art through routine experimentation.

The suitable desiccant material may be incorporated into the product package in any manner including a compressed pellet, or enclosed within a holder such as a capsule, sachet or container. Any material that is water vapor permeable and does not react with or adversely affect (for example, by leaching or absorption) components of the formulation or other materials used in making the product package is suitable for forming the desiccant holder. Such materials include polyethylene, polyethylene terephthalate, polypropylene, coated and non-coated paper, and perforated sheet and laminate materials. In one embodiment, the material for the desiccant holder may be non-woven polyolefin.

In further or alternative embodiments, the outer packaging contains oxygen-absorbing materials, in particular, when the outer packaging comprises a sealed container, such as a plastic bag or bottle. The oxygen-absorbing material can provide further protection to the solution Formula (I) formulations by absorbing keeping the level of oxygen surrounding the sealed container (i.e., the container which directly contains the Formula (I) formulation) to a minimum. Examples of oxygen-absorbing materials (also known as oxygen scavengers) include ascorbic acid, iron powder, and an inorganic ferrous salt such as ferrous-halide, -nitrate or -sulfide. Such oxygen-absorbing materials can be kept within an oxygen-permeable container, such as a sachet.

### Oxygen Absorber

In the pharmaceutical packaging systems described herein, oxygen absorbers absorb and remove oxygen from all components of the system. Oxygen absorbers are contemplated to be in any size or shape including sachet, pouch, capsule, label, strip, patch, cartridge, lining, sticker, etc., that is placed inside of the reservoir, but can also be integrated to the primary packaging. In some embodiments, the oxygen absorber is in the form of a capsule.

Suitable materials for oxygen absorbers include metal-based substances that remove oxygen by reacting with it by chemical bonding, generally forming a metal oxide component. Metal-based substances include elemental iron as well as iron oxide, iron hydroxide, iron carbide and the like. Other metals for use as oxygen absorbers include nickel, tin, copper and zinc. Metal-based oxygen absorbers are typically in the form of a powder to increase surface area. Powder formation of the metal-based oxygen absorbers is by any known method including, but not limited to, atomization, milling, pulverization, and electrolysis. Additional materials for oxygen absorbers include low molecular weight organic compounds such as ascorbic acid, sodium ascorbate, catechol and phenol, activated carbon and polymeric materials incorporating a resin and a catalyst. In some embodiments of the pharmaceutical packaging system, the oxygen absorber is a metal-based oxygen absorber. In certain instances of the pharmaceutical packaging system, the oxygen absorber is an iron-based oxygen absorber. In further instances of the pharmaceutical packaging system, the oxygen absorber is an iron-based oxygen absorber in the form of a canister.

Oxygen absorbents which can be used in the present invention include iron and glucose oxidase. A salt may be used as an electrolyte for oxidation of the iron. The iron may be hydrogen-reduced iron, electrolytically reduced iron, or chemically reduced iron. Although iron is preferred as the metallic oxygen absorbing agent, it will be appreciated that other metals may be used. These are, by way of example and not limitation, aluminum, copper, zinc, titanium, magnesium, and tin. Also, other elements which can be used in elemental or partially oxidized form are sodium, manganese, iodine, sulfur, and phosphorus.

The electrolytic salt may be sodium chloride or any other suitable food compatible salt including, but not limited to, sodium sulfate, potassium chloride, ammonium chloride, ammonium sulfate, calcium chloride, sodium phosphate, calcium phosphate, and magnesium chloride. An example of a suitable thermoplastic resin containing an oxygen absorber is Amosorb™ 3000 (available from BP Amoco Chemicals). Other resins appropriate for the current invention include those made using ascorbic acid or other easily oxidized organic compounds.

### Coloring Agents

Coloring agents can be used to color code the absorbent, for example, to indicate that the absorbent is not to be ingested. Suitable coloring agents include, without limitation, natural and/or artificial compounds such as FD&C coloring agents, natural juice concentrates, pigments such as titanium oxide, silicon dioxide, iron oxides, zinc oxide, combinations thereof, and the like.

Colorants/opacifiers for use in the present invention include organic dyes and their lakes, inorganic colors and natural colors, including water soluble colors and water-insoluble colors (pigments). The compositions of the present invention can comprise from about 0% to about 10% by weight of the flavoring and/or coloring agent, preferably from about 0.1% to about 5%, and more preferably from about 2% to about 3%.

### Packaging/Treatment Kits

The present invention relates to a kit for conveniently and effectively carrying out the methods in accordance with the present invention. Such kits may be suited for the delivery of solid oral forms such capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging pharmaceutical unit dosage forms. If desired, the blister can be in the form of a childproof blister, i.e., a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as an AM dose is packaged with a “mid day” and a PM dose.; or an AM dose is packaged with a PM dose. Alternatively, placebo dosages, or vitamin or dietary supplements, either in a form similar to or distinct from the pharmaceutical active dosages, can be included.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and a clear PVC. The PVC can be transparent so the item (pill, tablet, geltab, etc.) can be seen and examined easily; and in one aspect, can be vacuum-formed around a mold so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a pharmaceutical combination of the invention), and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or large.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

### Administration

The pharmaceutical compositions may be optimized for particular types of delivery. For example, pharmaceutical compositions for oral delivery are formulated using pharmaceutically acceptable carriers that are well known in the art. The carriers enable the agents in the composition to be formulated, for example, as a tablet, pill, capsule, solution, suspension, sustained release formulation; powder, liquid or gel for oral ingestion by the subject.

The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner

Typically, the composition may be applied repeatedly for a sustained period of time topically on the part of the body to be treated, for example, the eyelids, eyebrows, skin or scalp. The dosage regimen will generally involve regular, such as daily, administration for a period of treatment of at least one month, or at least three months, or at least six months.

Alternatively, the composition may be applied intermittently, or in a pulsed manner Accordingly, an alternative embodiment of the disclosure is to apply the composition on an intermittent or pulsed dosage schedule. For example, the composition of the disclosure may be used for two or more days, stopped, then restarted again at a time from between 2 weeks to 3 months later, and at even more long-spaced intervals in the case of the scalp.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Alternatively, the amount specified may be the amount administered as the average daily, average weekly, or average monthly dose.

The invention will be illustrated in more detail with reference to the following Examples, but it should be understood that the present invention is not deemed to be limited thereto.

### EXAMPLES

### Example 1

The below provided are the formulations prepared with BHA as antioxidant used for stabilizing Levothyroxine Sodium Tablets.

Composition of Levothyroxine Sodium Tablets containing BHA as antioxidant sufficient to inhibit oxidative degradation of drug in 0.01% of total weight.

Levothyroxine sodium tablets 300 mcg

Formulation 1

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.890 |
| BHA | 0.010 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA as antioxidant sufficient to inhibit oxidative degradation of drug in 0.05% of total weight.

Formulation 2

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.848 |
| BHA | 0.052 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA as antioxidant sufficient to inhibit oxidative degradation of drug in 0.1% of total weight.

Formulation 3

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.796 |
| BHA | 0.104 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA as antioxidant sufficient to inhibit oxidative degradation of drug in 0.15% of total weight.

Formulation 4

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.744 |
| BHA | 0.156 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA as antioxidant sufficient to inhibit oxidative degradation of drug in 0.2% of total weight.

Formulation 5

| Component | Qunatity in mg/tablet |
|---|---|
| Levothyroxine Sodium | 0.300 |
| Microcrystalline cellulose (PH 105) | 25.000 |
| Microcrystalline cellulose (PH 102) | 64.692 |
| BHA | 0.208 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA as antioxidant sufficient to inhibit oxidative degradation of drug in 0.25% of total weight.

Formulation 6

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.640 |
| BHA | 0.260 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA as antioxidant sufficient to inhibit oxidative degradation of drug in 0.30% of total weight.

Formulation 7

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.588 |
| BHA | 0.312 |
| Plasdone K 29/32 | 7.500 |
| Colloidal Silicon Dioxide | 1.000 |
| Colorants | 0.300 |
| Magnesium stearate | 1.000 |

Manufacturing Process

Levothyroxine sodium tablets were manufactured with above compositions via a direct compression process comprising the following steps:
1. Preparation of a Levothyroxine sodium triturate with BHA and microcrystalline cellulose PH 105 by geometric dilution method and sifted through 80# mesh.
2. The triturate is blended in blender for 30 mins.
3. Sift all other excipients Microcrystalline cellulose PH 102, Colloidal silicon dioxide, Povidone K 29/32 and colorants together through ASTM #60.
4. The step 2 mix is added to step 3 mix and blended in suitable blender for 60 mins. 5. Sift Magnesium stearate through ASTM #80 and lubricated with step 4 in blender for 5 min. 6. The final mix is compressed into tablets at a crushing strength of 7 kp. 7. Pack the tablets in a suitable container (Bottles or Blisters).

Stability Studies and Results

The compositions of above Example were formulated into tablets and introduced into suitable container for multiple dose.

Primary packing materials are HDPE containers with Child Resistant Caps.

They were placed under controlled conditions (temperature and humidity) to make the required stability studies.

The results of these stability studies are provided in Table 1 below

TABLE 1

| | Formulation | Formulation | Formulation | Formulation | Formulation | Formulation | Formulation |
|---|---|---|---|---|---|---|---|
| Parameter | 1 | 2 | 3 | 4 | 5 | 6 | 7 |

Assay (%)

| | | | | | | | |
|---|---|---|---|---|---|---|---|
| Initial | 99.6 | 101.3 | 99.0 | 102.3 | 98.0 | 97.7 | 99.7 |
| 40° C./75% | 98.3 | 100.9 | 97.6 | 100.2 | 97.6 | 96.6 | 97.5 |
| RH | | | | | | | |
| 1 Month | | | | | | | |
| 40° C./75% | 97.5 | 98.3 | 95.8 | 100.1 | 96.6 | 97.0 | 96.2 |
| RH | | | | | | | |
| 3 Months | | | | | | | |
| 40° C./75% | 97.2 | 98.0 | 95.6 | 99.5 | 96.2 | 96.6 | 96.0 |
| RH | | | | | | | |
| 6 Months | | | | | | | |

Impurities
Liothyronine (%)

| | | | | | | | |
|---|---|---|---|---|---|---|---|
| Initial | 0.0165 | 0.0172 | 0.0164 | 0.0147 | 0.0141 | 0.0157 | 0.0123 |
| 40° C./75% | 0.0087 | 0.0207 | 0.0224 | 0.0318 | 0.0375 | 0.0408 | ND |
| RH | | | | | | | |
| 1 Month | | | | | | | |
| 40° C./75% | 0.0107 | 0.0141 | 0.0157 | 0.0149 | 0.0128 | 0.0118 | 0.011 |
| RH | | | | | | | |
| 3 Months | | | | | | | |
| 40° C./75% | 0.0208 | 0.0420 | 0.0357 | 0.029 | 0.026 | 0.035 | 0.048 |
| RH | | | | | | | |
| 6 Months | | | | | | | |

Total degradation products (%)

| | | | | | | | |
|---|---|---|---|---|---|---|---|
| Initial | 0.4782 | 0.4521 | 0.4077 | 0.3623 | 0.4079 | 0.4887 | 0.3104 |
| 40° C./75% | 0.604 | 0.565 | 0.5621 | 0.589 | 0.5322 | 0.5867 | 0.217 |
| RH | | | | | | | |
| 1 Month | | | | | | | |
| 40° C./75% | 0.5465 | 0.5884 | 0.6474 | 0.6434 | 0.5682 | 0.604 | 0.565 |
| RH | | | | | | | |
| 3 Months | | | | | | | |
| 40° C./75% | 0.8107 | 0.9141 | 0.7157 | 0.9149 | 0.8128 | 0.8118 | 0.911 |
| RH | | | | | | | |
| 6 Months | | | | | | | |

### Example 2

The below provided are the formulations prepared with BHT as antioxidant used for stabilizing Levothyroxine Sodium Tablets.

Composition of Levothyroxine Sodium Tablets containing BHT as antioxidant sufficient to inhibit oxidative degradation of drug in 0.01% of total weight.

Levothyroxine sodium tablets 300 mcg

Formulation 8

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.890 |
| BHT | 0.010 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHT as antioxidant sufficient to inhibit oxidative degradation of drug in 0.05% of total weight.

Formulation 9

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.848 |
| BHT | 0.052 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHT as antioxidant sufficient to inhibit oxidative degradation of drug in 0.1% of total weight.

Formulation 10

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.796 |
| BHT | 0.104 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHT as antioxidant sufficient to inhibit oxidative degradation of drug in 0.15% of total weight.

Formulation 11

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.744 |
| BHT | 0.156 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHT as antioxidant sufficient to inhibit oxidative degradation of drug in 0.2% of total weight.

Formulation 12

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.692 |
| BHT | 0.208 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHT as antioxidant sufficient to inhibit oxidative degradation of drug in 0.25% of total weight.

Formulation 13

| Component | Quantity in mg/tablet |
|---|---|
| Levothyroxine Sodium | 0.300 |
| Microcrystalline cellulose (PH 105) | 25.000 |
| Microcrystalline cellulose (PH 102) | 64.640 |
| BHT | 0.260 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHT as antioxidant sufficient to inhibit oxidative degradation of drug in 0.30% of total weight.

Formulation 14

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.588 |
| BHT | 0.312 |
| Plasdone K 29/32 | 7.500 |
| Colloidal Silicon Dioxide | 1.000 |
| Colorants | 0.300 |
| Magnesium stearate | 1.000 |

Manufacturing Process

Levothyroxine sodium tablets were manufactured with above compositions via a direct compression process comprising the following steps:
1. Preparation of a Levothyroxine sodium triturate with BHT and microcrystalline cellulose PH 105 by geometric dilution method and sifted through 80# mesh.
2. The triturate is blended in blender for 30 mins.
3. Sift all other excipients Microcrystalline cellulose PH 102, Colloidal silicon dioxide, Povidone K 29/32 and colorants together through ASTM #60.
4. The step 2 mix is added to step 3 mix and blended in suitable blender for 60 mins.
5. Sift Magnesium stearate through ASTM #80 and lubricated with step 4 in blender for 5 min.
6. The final mix is compressed into tablets at a crushing strength of 7 kp.
7. Pack the tablets in a suitable container (Bottles or Blisters).

Stability Studies and Results

The compositions of above Example were formulated into tablets and introduced into suitable container for multiple dose.

Primary packing materials are HDPE containers with Child Resistant Caps.

They were placed under controlled conditions (temperature and humidity) to make the required stability studies.

The results of these stability studies are provided in Table 2 below

TABLE 2

| | Formulation | Formulation | Formulation | Formulation | Formulation | Formulation | Formulation |
|---|---|---|---|---|---|---|---|
| Parameter | 8 | 9 | 10 | 11 | 12 | 13 | 14 |

Assay (%)

| | | | | | | | |
|---|---|---|---|---|---|---|---|
| Initial | 98.0 | 98.7 | 99.7 | 99.5 | 99.3 | 99.7 | 99.0 |
| 40° C./75% | 97.6 | 96.6 | 99.5 | 97.6 | 96.6 | 97.5 | 97.8 |
| RH | | | | | | | |
| 1 Month | | | | | | | |
| 40° C./75% | 96.6 | 97.0 | 98.2 | 96.5 | 96.3 | 97.0 | 97.2 |
| RH | | | | | | | |
| 3 Months | | | | | | | |
| 40° C./75% | 96.2 | 96.7 | 96.9 | 95.8 | 95.7 | 96.6 | 96.3 |
| RH | | | | | | | |
| 6 Months | | | | | | | |

Impurities
Liothyronine (%)

| | | | | | | | |
|---|---|---|---|---|---|---|---|
| Initial | 0.0157 | 0.0123 | 0.0148 | 0.0157 | 0.0241 | 0.0164 | 0.0147 |
| 40° C./75% | 0.0318 | 0.0375 | 0.0318 | 0.0318 | 0.0375 | 0.0408 | 0.0356 |
| RH | | | | | | | |
| 1 Month | | | | | | | |
| 40° C./75% | 0.0352 | 0.0466 | 0.0337 | 0.0349 | 0.0428 | 0.0435 | 0.0412 |
| RH | | | | | | | |
| 3 Months | | | | | | | |
| 40° C./75% | 0.0488 | 0.0520 | 0.0572 | 0.0585 | 0.0567 | 0.0651 | 0.0483 |
| RH | | | | | | | |
| 6 Months | | | | | | | |

Total degradation products (%)

| | | | | | | | |
|---|---|---|---|---|---|---|---|
| Initial | 0.4028 | 0.4217 | 0.4703 | 0.4423 | 0.4237 | 0.4733 | 0.4310 |
| 40° C./75% | 0.5465 | 0.5884 | 0.6474 | 0.6434 | 0.5682 | 0.6040 | 0.565 |
| RH | | | | | | | |
| 1 Month | | | | | | | |
| 40° C./75% | 0.6538 | 0.6884 | 0.6671 | 0.6934 | 0.6628 | 0.6954 | 0.7265 |
| RH | | | | | | | |
| 3 Months | | | | | | | |
| 40° C./75% | 0.7456 | 0.8141 | 0.7157 | 0.8492 | 0.8238 | 0.8853 | 0.8415 |
| RH | | | | | | | |
| 6 Months | | | | | | | |

### Example 3

The below provided are the formulations prepared with BHA & BHT as combination of antioxidants used for stabilizing Levothyroxine Sodium Tablets.

Composition of Levothyroxine Sodium Tablets containing BHA & BHT as antioxidants sufficient to inhibit oxidative degradation of drug in 0.01% of total weight.

Levothyroxine sodium tablets 300 mcg

Formulation 15

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.890 |
| BHA | 0.005 |
| BHT | 0.005 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA & BHT as antioxidants sufficient to inhibit oxidative degradation of drug in 0.05% of total weight.

Formulation 16

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.848 |
| BHA | 0.026 |
| BHT | 0.026 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA & BHT as antioxidants sufficient to inhibit oxidative degradation of drug in 0.1% of total weight.

Formulation 17

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.796 |
| BHA | 0.052 |
| BHT | 0.052 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA & BHT as antioxidants sufficient to inhibit oxidative degradation of drug in 0.15% of total weight.

Formulation 18

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.744 |
| BHA | 0.078 |
| BHT | 0.078 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA & BHT as antioxidants sufficient to inhibit oxidative degradation of drug in 0.2% of total weight.

Formulation 19

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.692 |
| BHA | 0.104 |
| BHT | 0.104 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA & BHT as antioxidants sufficient to inhibit oxidative degradation of drug in 0.25% of total weight.

Formulation 20

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.640 |
| BHA | 0.130 |
| BHT | 0.130 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing BHA & BHT as antioxidants sufficient to inhibit oxidative degradation of drug in 0.30% of total weight.

Formulation 21

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.588 |
| BHA | 0.156 |
| BHT | 0.156 |
| Plasdone K 29/32 | 7.500 |
| Colloidal Silicon Dioxide | 1.000 |
| Colorants | 0.300 |
| Magnesium stearate | 1.000 |

Manufacturing Process

Levothyroxine sodium tablets were manufactured with above compositions via a direct compression process comprising the following steps:
1. Preparation of a Levothyroxine sodium triturate with BHA & BHT and microcrystalline cellulose PH 105 by geometric dilution method and sifted through 80# mesh.
2. The triturate is blended in blender for 30 mins.
3. Sift all other excipients Microcrystalline cellulose PH 102, Colloidal silicon dioxide, Povidone K 29/32 and colorants together through ASTM #60.
4. The step 2 mix is added to step 3 mix and blended in suitable blender for 60 mins.
5. Sift Magnesium stearate through ASTM #80 and lubricated with step 4 in blender for 5 min.
6. The final mix is compressed into tablets at a crushing strength of 7 kp.
7. Pack the tablets in a suitable container (Bottles or Blisters).

Stability Studies and Results

The compositions of above Example were formulated into tablets and introduced into suitable container for multiple dose.

Primary packing materials are HDPE containers with Child Resistant Caps.

They were placed under controlled conditions (temperature and humidity) to make the required stability studies.

The results of these stability studies are provided in Table 3 below

TABLE 3

| | Formulation | Formulation | Formulation | Formulation | Formulation | Formulation | Formulation |
|---|---|---|---|---|---|---|---|
| Parameter | 15 | 16 | 17 | 18 | 19 | 20 | 21 |

Assay ( %)

| | | | | | | | |
|---|---|---|---|---|---|---|---|
| Initial | 99.9 | 99.2 | 100.0 | 100.3 | 99.0 | 99.7 | 99.5 |
| 40° C./75% | 98.5 | 98.3 | 97.8 | 100.1 | 98.6 | 98.0 | 98.2 |
| RH | | | | | | | |
| 1 Month | | | | | | | |
| 40° C./75% | 97.4 | 97.6 | 96.9 | 99.7 | 97.3 | 97.2 | 96.7 |
| RH | | | | | | | |
| 3 Months | | | | | | | |
| 40° C./75% | 97.2 | 97.0 | 96.6 | 98.5 | 96.2 | 96.0 | 96.5 |
| RH | | | | | | | |
| 6 Months | | | | | | | |

Impurities
Liothyronine (%)

| | | | | | | | |
|---|---|---|---|---|---|---|---|
| Initial | 0.0107 | 0.0141 | 0.0157 | 0.0149 | 0.0128 | 0.0118 | 0.011 |
| 40° C./75% | 0.0157 | 0.0149 | 0.0175 | 0.0218 | 0.0267 | 0.0235 | 0.0149 |
| RH | | | | | | | |
| 1 Month | | | | | | | |
| 40° C./75% | 0.0307 | 0.0341 | 0.0357 | 0.0249 | 0.0293 | 0.0282 | 0.0310 |
| RH | | | | | | | |
| 3 Months | | | | | | | |
| 40° C./75% | 0.0508 | 0.0420 | 0.0573 | 0.0495 | 0.0364 | 0.0476 | 0.0487 |
| RH | | | | | | | |
| 6 Months | | | | | | | |

Total degradation products (%)

| | | | | | | | |
|---|---|---|---|---|---|---|---|
| Initial | 0.5682 | 0.6046 | 0.5653 | 0.5623 | 0.5465 | 0.5884 | 0.6474 |
| 40° C./75% | 0.6455 | 0.6534 | 0.5875 | 0.689 | 0.5924 | 0.6678 | 0.7178 |
| RH | | | | | | | |
| 1 Month | | | | | | | |
| 40° C./75% | 0.7653 | 0.8853 | 0.7443 | 0.8345 | 0.8627 | 0.8456 | 0.8544 |
| RH | | | | | | | |
| 3 Months | | | | | | | |
| 40° C./75% | 0.9704 | 0.9451 | 0.9567 | 0.9459 | 0.9238 | 0.9718 | 0.9811 |
| RH | | | | | | | |
| 6 Months | | | | | | | |

### Example 4

The below provided are the formulations prepared with Vitamin C as antioxidant used for stabilizing Levothyroxine Sodium Tablets.

Composition of Levothyroxine Sodium Tablets containing Vitamin C as antioxidant sufficient to inhibit oxidative degradation of drug in 0.01% of total weight.

Levothyroxine sodium tablets 300 mcg

Formulation 1

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.890 |
| Vitamin C | 0.010 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing Vitamin C as antioxidant sufficient to inhibit oxidative degradation of drug in 0.05% of total weight.

Formulation 2

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.848 |
| Vitamin C | 0.052 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing Vitamin C as antioxidant sufficient to inhibit oxidative degradation of drug in 0.1% of total weight.

Formulation 3

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.796 |
| Vitamin C | 0.104 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing Vitamin C as antioxidant sufficient to inhibit oxidative degradation of drug in 0.15% of total weight.

Formulation 4

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.744 |
| Vitamin C | 0.156 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing Vitamin C as antioxidant sufficient to inhibit oxidative degradation of drug in 0.2% of total weight.

Formulation 5

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.692 |
| Vitamin C | 0.208 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing Vitamin C as antioxidant sufficient to inhibit oxidative degradation of drug in 0.25% of total weight.

Formulation 6

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.640 |
| Vitamin C | 0.260 |
| Plasdone K 29/32 | 7.500 |
| Colloidal Silicon Dioxide | 1.000 |
| Colorants | 0.300 |
| Magnesium stearate | 1.000 |

Composition of Levothyroxine Sodium Tablets containing Vitamin C as antioxidant sufficient to inhibit oxidative degradation of drug in 0.30% of total weight.

Formulation 7

| | Quantity in |
|---|---|
| Component | mg/tablet |
| Levothyroxine Sodium | 0.300 |
| Microcrystalline cellulose (PH 105) | 25.000 |
| Microcrystallline cellulose (PH 102) | 64.588 |
| Vitamin C | 0.312 |
| Plasdone K 29/32 | 7.500 |
| Colloidal Silicon Dioxide | 1.000 |
| Colorants | 0.300 |
| Magnesium stearate | 1.000 |

Manufacturing Process

Levothyroxine sodium tablets were manufactured with above compositions via a direct compression process comprising the following steps:
1. Preparation of a Levothyroxine sodium triturate with Vitamin C and microcrystalline cellulose PH 105 by geometric dilution method and sifted through 80# mesh.
2. The triturate is blended in blender for 30 mins.
3. Sift all other excipients Microcrystalline cellulose PH 102, Colloidal silicon dioxide, Povidone K 29/32 and colorants together through ASTM #60.
4. The step 2 mix is added to step 3 mix and blended in suitable blender for 60 mins.
5. Sift Magnesium stearate through ASTM #80 and lubricated with step 4 in blender for 5 min.
6. The final mix is compressed into tablets at a crushing strength of 7 kp.
7. Pack the tablets in a suitable container (Bottles or Blisters).

Stability Studies and Results

The compositions of above Example were formulated into tablets and introduced into suitable container for multiple dose.

Primary packing materials are HDPE containers with Child Resistant Caps.

They were placed under controlled conditions (temperature and humidity) to make the required stability studies.

The results of these stability studies are provided in Table 4 below

TABLE 4

| | Formulation | Formulation | Formulation | Formulation | Formulation | Formulation | Formulation |
|---|---|---|---|---|---|---|---|
| Parameter | 22 | 23 | 24 | 25 | 26 | 27 | 28 |

Assay (%)

| | | | | | | | |
|---|---|---|---|---|---|---|---|
| Initial | 100.6 | 99.3 | 99.8 | 100.3 | 99.5 | 98.8 | 99.9 |
| 40° C./75% | 98.9 | 98.9 | 98.6 | 99.2 | 98.8 | 97.6 | 97.5 |
| RH | | | | | | | |
| 1 Month | | | | | | | |
| 40° C./75% | 97.2 | 98.0 | 96.8 | 98.1 | 97.6 | 97.0 | 96.7 |
| RH | | | | | | | |
| 3 Months | | | | | | | |
| 40° C./75% | 96.2 | 97.4 | 95.9 | 97.5 | 96.8 | 96.2 | 96.0 |
| RH | | | | | | | |
| 6 Months | | | | | | | |

Impurities
Liothyronine (%)

| | | | | | | | |
|---|---|---|---|---|---|---|---|
| Initial | 0.0155 | 0.0166 | 0.0158 | 0.0159 | 0.0146 | 0.0157 | 0.0139 |
| 40° C./75% | 0.0187 | 0.0228 | 0.0244 | 0.0358 | 0.0325 | 0.0425 | 0.0339 |
| RH | | | | | | | |
| 1 Month | | | | | | | |
| 40° C./75% | 0.0192 | 0.0241 | 0.0257 | 0.0349 | 0.0328 | 0.0418 | 0.0350 |
| RH | | | | | | | |
| 3 Months | | | | | | | |
| 40° C./75% | 0.0208 | 0.0320 | 0.0372 | 0.0353 | 0.0426 | 0.0435 | 0.0348 |
| RH | | | | | | | |
| 6 Months | | | | | | | |

Total degradation products (%)

| | | | | | | | |
|---|---|---|---|---|---|---|---|
| Initial | 0.4825 | 0.4219 | 0.4772 | 0.4623 | 0.4449 | 0.4987 | 0.4214 |
| 40° C./75% | 0.6470 | 0.5505 | 0.7612 | 0.7892 | 0.6325 | 0.7765 | 0.6172 |
| RH | | | | | | | |
| 1 Month | | | | | | | |
| 40° C./75% | 0.5465 | 0.5884 | 0.6474 | 0.6434 | 0.5682 | 0.6045 | 0.5650 |
| RH | | | | | | | |
| 3 Months | | | | | | | |
| 40° C./75% | 0.6178 | 0.7148 | 0.8478 | 0.8492 | 0.9285 | 0.8138 | 0.8280 |
| RH | | | | | | | |
| 6 Months | | | | | | | |

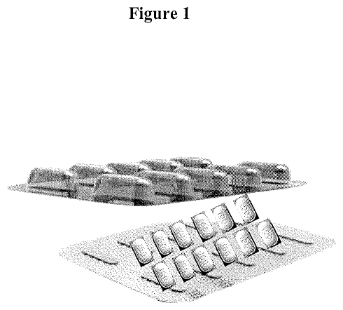

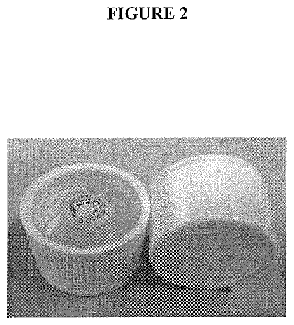

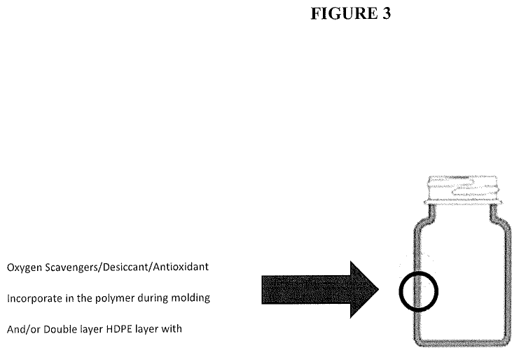

From the above formulations with different antioxidant combinations, formulations containing BHA as antioxidant in 0.1% of total weight were selected to carryout stability study in selective packing materials for further stabilization as below.
1. HDPE container with in wall desiccant as given in below FIG. 3.
2. A Child Resistant Cap of a High Density Polyethylene Bottle comprising an oxygen scavenger as given in below FIG. 2.
3. Blister sheet comprising of an array of interconnected cavities connected through channels that allows the exchange of air between them, wherein one or more of the cavities serve as a reservoir holding an oxygen scavenger as shown in FIG. 1.

While the invention has been described in detail and with reference to specific examples thereof, it will be apparent to one skilled in the art that various changes and modifications can be made therein without departing from the spirit and scope thereof.

What is claimed is:

1. A storage stable pharmaceutical tablet comprising an active agent which is sensitive to oxygen comprising:
a. A therapeutically effective amount of a thyroxine active drug; and
b. An amount of an antioxidant sufficient to inhibit oxidative degradation of active drug

2. The tablet in claim 1, wherein said thyroxine active drug is levothyroxine sodium.

3. The tablet in claim 1, wherein said antioxidant is selected from a group consisting of butyl hydroxyl anisole (BHA), butyl hydroxyl toluene (BHT), Vitamin C, and combinations thereof.

4. The tablet in claim 1, wherein said thyroxine active drug is comprised of levothyroxine sodium between 0.025 mg to about 0.3 mg.

5. The tablet in claim 1, wherein said amount of antioxidant sufficient to inhibit oxidative degradation of thyroxine active drug is 0.01% of total weight.

6. The tablet in claim 1, wherein said amount of antioxidant sufficient to inhibit oxidative degradation of thyroxine active drug is 0.05% of total weight.

7. The tablet in claim 1, wherein said amount of antioxidant sufficient to inhibit oxidative degradation of thyroxine active drug is 0.1% of total weight.

8. The tablet in claim 1, wherein said amount of antioxidant sufficient to inhibit oxidative degradation of thyroxine active drug is 0.15% of total weight.

9. The tablet in claim 1, wherein said amount of antioxidant sufficient to inhibit oxidative degradation of thyroxine active drug is 0.2% of total weight.

10. The tablet in claim 1, wherein said amount of antioxidant sufficient to inhibit oxidative degradation of thyroxine active drug is 0.25% of total weight.

11. The tablet in claim 1, wherein said amount of antioxidant sufficient to inhibit oxidative degradation of drug is 0.3% of total weight.

12. A composition of claim 1 wherein said antioxidant is BHA.

13. A storage stable pharmaceutical tablet comprising an active agent that is sensitive to oxygen comprising:
a. A therapeutically effective amount of a thyroxine active drug; and
b. An amount of an antioxidant sufficient to inhibit oxidative degradation of active drug.

14. The tablet in claim 13, wherein said thyroxine active drug is levothyroxine sodium.

15. The tablet in claim 13 wherein said antioxidant is selected from a group consisting of BHA, BHT and Vitamin C.

16. The tablet in claim 13, wherein said thyroxine active drug is comprised of levothyroxine sodium between 0.025 mg to about 0.3 mg.

17. The tablet in claim 13, wherein said amount of antioxidant sufficient to inhibit oxidative degradation of thyroxine active drug is 0.01% of total weight.

18. The tablet in claim 13, wherein said amount of antioxidant sufficient to inhibit oxidative degradation of thyroxine active drug is 0.05% of total weight.

19. The tablet in claim 13, wherein said amount of antioxidant sufficient to inhibit oxidative degradation of thyroxine active drug is 0.1% of total weight.

20. The tablet in claim 13, wherein said amount of antioxidant sufficient to inhibit oxidative degradation of thyroxine active drug is 0.15% of total weight.

21. The tablet in claim 13, wherein said amount of antioxidant sufficient to inhibit oxidative degradation of thyroxine active drug is 0.2% of total weight.

22. The tablet in claim 13, wherein said amount of antioxidant sufficient to inhibit oxidative degradation of thyroxine active drug is 0.25% of total weight.

23. The tablet in claim 13, wherein said amount of antioxidant sufficient to inhibit oxidative degradation of thyroxine active drug is 0.3% of total weight.

24. A composition of claim 13 wherein said antioxidant is BHT.

25. A storage stable pharmaceutical tablet comprising an active agent that is sensitive to oxygen comprising:
a. A therapeutically effective amount of a thyroxine active drug; and
b. An amount of an antioxidant sufficient to inhibit oxidative degradation of active drug.

26. The tablet in claim 25, wherein said thyroxine active drug is levothyroxine sodium.

27. The tablet in claim 25 wherein said antioxidant is selected from a group consisting of BHA, BHT and Vitamin C.

28. The tablet in claim 25, wherein said active drug is comprised of levothyroxine sodium between 0.025 mg to about 0.3 mg.

29. The tablet in claim 25, wherein said amount of antioxidant sufficient to inhibit oxidative degradation of thyroxine active drug is 0.01% of total weight.

30. The tablet in claim 25, wherein said amount of antioxidant sufficient to inhibit oxidative degradation of thyroxine active drug is 0.05% of total weight.

31. The tablet in claim 25, wherein said amount of antioxidant sufficient to inhibit oxidative degradation of thyroxine active drug is 0.1% of total weight.

32. The tablet in claim 25, wherein said amount of antioxidant sufficient to inhibit oxidative degradation of thyroxine active drug is 0.15% of total weight.

33. The tablet in claim 25, wherein said amount of antioxidant sufficient to inhibit oxidative degradation of thyroxine active drug is 0.2% of total weight.

34. The tablet in claim 25, wherein said amount of antioxidant sufficient to inhibit oxidative degradation of thyroxine active drug is 0.25% of total weight.

35. The tablet in claim 25, wherein said amount of antioxidant sufficient to inhibit oxidative degradation of thyroxine active drug is 0.3% of total weight.

36. A composition of claim 25 wherein said antioxidant is Vitamin C.

37. A storage stable pharmaceutical tablet comprising an active agent that is sensitive to oxygen comprising:
a. A therapeutically effective amount of a thyroxine active drug; and
b. An amount of an antioxidant sufficient to inhibit oxidative degradation of active drug.

38. The tablet in claim 37, wherein said thyroxine active drug is levothyroxine sodium.

39. The tablet in claim 37 wherein said antioxidant is selected from a group consisting of BHA, BHT and Vitamin C.

40. The tablet in claim 37, wherein said active drug is comprised of levothyroxine sodium between 0.025 mg to about 0.3 mg.

41. The tablet in claim 37, wherein said amount of antioxidant is sufficient to inhibit oxidative degradation of thyroxine active drug is 0.005% and 0.005% of total weight.

42. The tablet in claim 37, wherein said amount of antioxidant is sufficient to inhibit oxidative degradation of thyroxine active drug is 0.025% and 0.025% of total weight.

43. The tablet in claim 37, wherein said amount of antioxidant is sufficient to inhibit oxidative degradation of thyroxine active drug is 0.05% and 0.05% of total weight.

44. The tablet in claim 37, wherein said amount of antioxidant is sufficient to inhibit oxidative degradation of thyroxine active drug is 0.075% and 0.075% of total weight.

45. The tablet in claim 37, wherein said amount of antioxidant is sufficient to inhibit oxidative degradation of thyroxine active drug is 0.1% and 0.1% of total weight.

46. The tablet in claim 37, wherein said amount of antioxidant is sufficient to inhibit oxidative degradation of thyroxine active drug is 0.125% and 0.125% of total weight.

47. The tablet in claim 37, wherein said amount of antioxidant is sufficient to inhibit oxidative degradation of thyroxine active drug is 0.15% and 0.15% of total weight.

48. A composition of claim 37 wherein said antioxidant is BHA and BHT in equal amounts.

49. A packaging bottle for a pharmaceutical product comprising an active agent that is sensitive to oxygen comprising a high density polyethylene bottle comprising an antioxidant sufficient to inhibit oxidative degradation

50. The package in claim 49, wherein the pharmaceutical product contains an active agent comprising levothyroxine sodium.

51. The package in claim 49, wherein said antioxidants are BHA, BHT and Vitamin C.

52. A packaging cap for a pharmaceutical product comprising an active agent that is sensitive to oxygen and/or moisture comprising:
a. A Child-Resistant Cap of a High Density Polyethylene Bottle comprising:
i. An oxygen scavenger and/or a desiccant.

53. The package in claim 52, wherein the pharmaceutical product contains an active agent comprising of levothyroxine sodium.

54. The package in claim 52, wherein the oxygen scavenger is selected from the group consisting of organic, inorganic, metallic, non-metallic and enzymatic in nature.

55. The package in claim 52, wherein the desiccant is selected from the group consisting of organic desiccants, inorganic desiccants, uncoated polymers, coated adsorbent polymers, and combinations thereof.

56. A packaging system for a pharmaceutical product comprising an active agent that is sensitive to oxygen comprising:
a. A blister pack comprising:
i. A blister sheet comprising of an array of interconnected cavities connected through channels that allows the exchange of air between them;
ii. Wherein one or more of the cavities serve as a reservoir holding an oxygen scavenger;
iii. Further wherein one or more of the cavities comprising at least one pharmaceutical product that is sensitive to oxygen; and
iv. A frangible lidding sealed to the sheet protecting the product in the cavity.

57. The package in claim 56, wherein the pharmaceutical product contains an active agent that comprises levothyroxine sodium.

58. The package in claim 56, wherein the blister sheet has light and moisture protectant properties.

59. The package in claim 56 wherein the blister sheet is made of materials selected from the group consisting of polyvinylchloride, polyvinylidene chloride, polycarbonate, polyester, copolyester, acrylonitrile, low density polyethylene, polypropylene, and combinations thereof.

60. The package in claim 56 wherein the oxygen scavenger is selected from the group consisting of an organic, inorganic, metallic, non-metallic, and enzymatic in nature.

61. The package in claim 56 wherein the frangible lidding has moisture barrier properties.
